(19) United States
(12) Patent Application Publication
(10) Pub. No.: US 2013/0109748 A1
(43) Pub. Date: May 2, 2013

(54) METHOD FOR OBTAINING EXTRACT OF CALLIGONUM SPECIES AND EXTRACT THEREOF

(71) Applicants: Dr. Nasser Saleh Ali Al-Khalifah, Riyadh (SA); Shanavaskhan A.E., Riyadh (SA)

(72) Inventors: Dr. Nasser Saleh Ali Al-Khalifah, Riyadh (SA); Shanavaskhan A.E., Riyadh (SA)

(73) Assignee: King Abdul Aziz City for Science and Technology, Riyadh (SA)

(21) Appl. No.: 13/210,428

(22) Filed: Aug. 16, 2011

(51) Int. Cl. A61K 31/352

(52) U.S. Cl. CPC A61K 31/352
USPC 514/456

(57) ABSTRACT

A method for obtaining a plurality of extracts from Calligonum sp. is disclosed. The method includes boiling one or more stalks of Calligonum sp., along with one or more liquids in a sterilizing apparatus for a first predefined time duration at a predefined temperature and a predefined pressure. Thereafter, a plurality of extracts from the one or more stalks of Calligonum sp., are obtained. The plurality of extracts includes one or more sterile liquid extracts and one or more sterile residues. The one or more sterile liquid extracts and the one or more sterile residues are separated, wherein concentration of one or more active ingredients in the one or more sterile liquid extracts is greater as compared with one or more active ingredients in the extracts of Calligonum sp. obtained by conventional methods of extraction.

### FIELD OF THE INVENTION

The invention generally relates to a method for obtaining an extract from plants. More specifically, the invention relates to a method for obtaining a sterile extract of Calligonum sp and extract thereof.

### BACKGROUND OF THE INVENTION

Typically, decoction of extract from herbal plants is done using traditional techniques. The extract from the herbal plants includes chemical compounds having therapeutic properties. Traditional techniques include the boiling of one or more of a root, a stem, or a leaf of the herbal plants. The one or more roots, stems or leaves may be boiled in an open container such as a beaker, or a kettle using an electric heater, a gas heater, or an open fire. However, in these techniques the process of boiling is slow and time consuming. Further, it is a challenge to control temperature, pressure and duration of extraction while using traditional techniques. Accordingly, composition of the chemical constituents in the extract may vary based on the applied temperature and pressure, and the duration for which decoction of the herbal plants is performed. Further, the extract is contaminated easily with bacterial and fungal spores as a result of impurities present in the extract.

Therefore, there is a need in the art for a method for controlling temperature, pressure, and boiling time for obtaining a sterile extract of herbal plants.

### BRIEF DESCRIPTION OF THE FIGURES

FIG. 1 illustrates a flow chart of a method for extracting a plurality of extracts from Calligonum sp. in accordance with an embodiment of the invention.

FIG. 2 illustrates a chromatogram for the extract in accordance with Example 1.

FIG. 3 illustrates a chromatogram for the extract in accordance with a conventional method used to obtain the extract in accordance with Example 2.

### DETAILED DESCRIPTION OF THE INVENTION

Before describing in detail embodiments that are in accordance with the invention, it should be observed that the embodiments reside primarily in combinations of method steps and apparatus components related to methods for obtaining a sterile extract of Calligonum sp. Accordingly, the apparatus components and method steps have been represented where appropriate by conventional symbols in the drawings, showing only those specific details that are pertinent to understanding the embodiments of the invention so as not to obscure the disclosure with details that will be readily apparent to those of ordinary skill in the art having the benefit of the description herein.

In this document, relational terms such as first and second, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, or apparatus that comprises the element.

Generally speaking, pursuant to various embodiments, the invention provides a method for extracting a plurality of extracts from Calligonum sp. The method includes boiling one or more stalks of Calligonum sp., along with one or more liquids in a sterilizing apparatus for a first predefined time duration at a predefined temperature and a predefined pressure. Upon boiling, the plurality of extracts are obtained from the one or more stalks of Calligonum sp. The plurality of extracts includes one or more sterile liquid extracts and one or more sterile residues. The one or more sterile liquid extracts are separated from the one or more sterile residues. Concentration of one or more active ingredients in the one or more sterile liquid extracts is greater as compared to concentration of extracts of Calligonum sp. obtained using conventional methods of extraction.

In accordance with various embodiments, the one or more stalks of Calligonum sp. were collected from field growing Calligonum sp. plants. Calligonum is a genus of the family polygonaceae. Approximately 80 species of Calligonum are found in Asia and the Mediterranean region. The Calligonum sp., may include but are not limited to one of a Calligonum comosum, Calligonum crinitum and Calligonum tetrapteurum. The one or more stalks are washed with running water. Thereafter, the one or more stalks are cut uniformly. The one or more stalks are then placed inside a heat resistant bag. It will be apparent to a person skilled in the art that the heat resistant bag is not limited to one or more of an autoclaving bag, a polypropylene bag, and a conical flask but may include other means and combinations thereof known in the art.

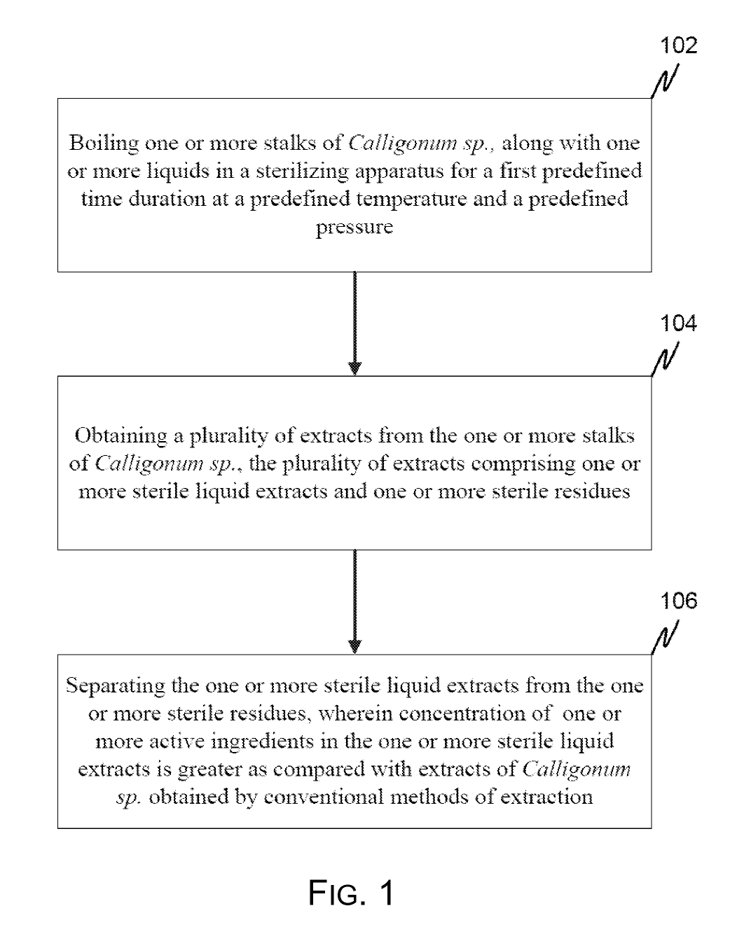

FIG. 1 illustrates a flow chart of a method for extracting a plurality of extracts from Calligonum sp. in accordance with an embodiment of the invention. The one or more stalks present in a heat resistant bag are boiled along with one or more liquids in a sterilizing apparatus for a first predefined time duration at a predefined temperature and a predefined pressure at step 102. The one or more liquids may include but is not limited to water. The one or more fluids at a predetermined volume may be present in the heat resistant bag. The predetermined volume of water ranges from 500 ml to 1000 ml in the heat resistant bag. The one or more fluids may be added per 0.25 kg to 0.75 kg of the one or more stalks in the heat resistant bag. The heat resistant bag is placed inside a sterilizing apparatus. The one or more stalks are boiled in the presence of steam in the sterilizing apparatus at the predefined temperature and the predefined pressure. The sterilizing apparatus includes, but is not limited to one of an autoclave, a pressure cooker, a closed container, and a bioreactor. The predefined temperature within the sterilizing apparatus ranges from 100° C. to 150° C. Further, the predefined pressure ranges from 10 psi to 20 psi. The first predefined time duration for boiling the at least one stalk ranges from 20 minutes to 40 minutes. At step 104, a plurality of extracts from the one or more stalks of Calligonum sp., are obtained. The plurality of extracts includes one or more sterile liquid extracts and one or more sterile residues.

Thereafter, at step 106, the one or more sterile liquid extracts are separated from the one or more sterile residues. A concentration of one or more active ingredients in the one or more sterile liquid extracts is greater as compared with one or more active ingredients in the extracts of Calligonum sp. obtained by conventional methods of extraction. The one or more active ingredients may include but are not limited to one of a flavonoid, a kaempferol, a quercetin, a quercetin-3-0-β-D-glycoside (isoquercitin), a kaempferol-3-0-β-glucuronide, a catechin, a rutin, a procyanidin, a carotinoid, a violaxanthin, a neoxanthin tannin, and a sterol or a triterpene. In an embodiment, the concentration of the one or more active ingredients such as, quercetin is greater as compared with one or more active ingredients in the extracts of Calligonum sp. obtained by conventional methods of extraction. The concentration of quercetin obtained ranges from about 15 ppm to about 17 ppm. The one or more active ingredients in the one or more sterile liquid extracts may include one of an anti-inflammatory, an antioxidant, an antimicrobial, an anticancer, a cardio-protective, a neuro-protective, an antidiabetic, an anti-osteoporotic, an estrogenic/anti-estrogenic, an anxiolytic, an analgesic, an anti-allergic an anti-ulcer and a cytoprotective ingredient.

Further, the one or more sterile residues present in the plurality of extracts are transferred to one of a paper bag, a petri dish, and a beaker. These one or more sterile residues are dried at a predefined temperature for a second predefined time duration in an oven. The oven may include, but is not limited to a hot air oven, a vacuum oven, and a drying oven. Alternatively, the one or more sterile residues may be dried under shade for duration of two days to four days. The predefined temperature for drying the one or more sterile residues ranges from 40° C. to 60° C. The second predefined time duration for drying the one or more sterile residues ranges from 24 hours to 96 hours.

In an aspect of the invention, an extract from Calligonum sp., is obtained using a method as explained in conjunction with FIG. 1. One or more stalks of Calligonum sp. were collected from field growing Calligonum sp. plants. The Calligonum sp., include but are not limited to one of a Calligonum comosum, Calligonum crinitum and Calligonum tetrapteurum. The one or more stalks are washed with running water. Thereafter, the one or more stalks are cut uniformly. The one or more stalks are then placed inside a heat resistant bag. The one or more stalks in the heat resistant bag are boiled along with one or more liquids in a sterilizing apparatus for a first predefined time duration at a predefined temperature and a predefined pressure. The predefined temperature ranges from 100° C. to 150° C. The predefined pressure ranges from 10 psi to 20 psi. The first predefined time duration for boiling the at least one stalk ranges from 20 minutes to 40 minutes. The heat resistant bag is placed inside a sterilizing apparatus. The sterilizing apparatus includes, but not limited to an autoclave, a pressure cooker, a closed container, and a bioreactor.

Subsequently, one or more sterile liquid extracts is obtained from the one or more stalks of Calligonum sp. The concentration of one or more active ingredients in the one or more sterile liquid extracts is greater as compared with extracts of Calligonum sp. obtained by conventional methods of extraction. The one or more active ingredients may include but are not limited to one of a flavonoid, a kaempferol, a quercetin, a quercetin-3-0-β-D-glycoside (isoquercitin), a kaempferol-3-0-β-glucuronide, a catechin, a rutin, a procyanidin, a carotinoid, a violaxanthin, a neoxanthin tannin, and a sterol or a triterpene. In an embodiment, the concentration of the one or more active ingredients such as quercetin is greater as compared with one or more active ingredients in the extracts of Calligonum sp. obtained by conventional methods of extraction. The concentration of quercetin thus obtained ranges from about 15 ppm to about 17 ppm. The one or more active ingredients in the one or more sterile liquid extracts may include one of an anti-inflammatory, an antioxidant, an antimicrobial, an anticancer, a cardio-protective, a neuro-protective, an antidiabetic, an anti-osteoporotic, an estrogenic/anti-estrogenic, an anxiolytic, an analgesic, an anti-allergic an anti-ulcer and a cytoprotective ingredient.

### Example 1

A method of extracting a plurality of extracts from Calligonum comosum.

Young green stalks of Calligonum comosum were collected from a field growing Calligonum comosum plants. These young green stalks were washed thoroughly with running water. The green stalks are then cut into a size of one inch using a sharp cutter. About 500 gms of cut stalks are then placed inside an autoclaving bag. Thereafter, 1000 ml of distil water was added to the autoclaving bag. Subsequently, the autoclaving bag was placed inside a basket of a Hirayama hiclave vertical autoclave HV110. For autoclaving, the temperature of the autoclave was maintained at 121° C. and pressure was maintained at 15 psi for a duration of 20 minutes. Thereafter, the autoclave is opened once the autoclave reached a temperature of 60-65° C. Subsequently, the autoclaving bag is removed from the autoclave. The extract of Calligonum comosum is obtained from the autoclaving bag by piercing a hole at the bottom of the autoclaving bag. The extract is filtered using a sterile sieve and collected in pre sterilized closed containers. The closed containers are stored at 25° C. The residue of the young green stalks remaining in the autoclaving bag is transferred to a paper bag. The residue is then dried in an electric oven at 40° C. for 72 hours. The volume of the extract obtained was 850 ml and was not contaminated.

### Comparative Example 2

A conventional method of extracting a plurality of extracts from Calligonum comosum.

Young green stalks of Calligonum comosum were collected from a field growing Calligonum comosum plants. The young green stalks were washed thoroughly with running water. The green stalks are then cut into a size of one inch using a sharp cutter. About 500 gms of cut stalks are then placed inside a 2 liter glass beaker. Thereafter, 1000 ml of distilled water was added to the glass beaker. The glass beaker along with the distilled water and the green stalks was placed on an electric heater for duration of 30 minutes. Thereafter, the glass beaker was cooled and the extract was filtered and stored in sterilized bottles at 25° C. The volume of extract obtained using a conventional method was found to be 700 ml and contaminated after a period of 7 days.

### Example 3

Analysis of the extracts obtained using methods described in example 1 and example 2.

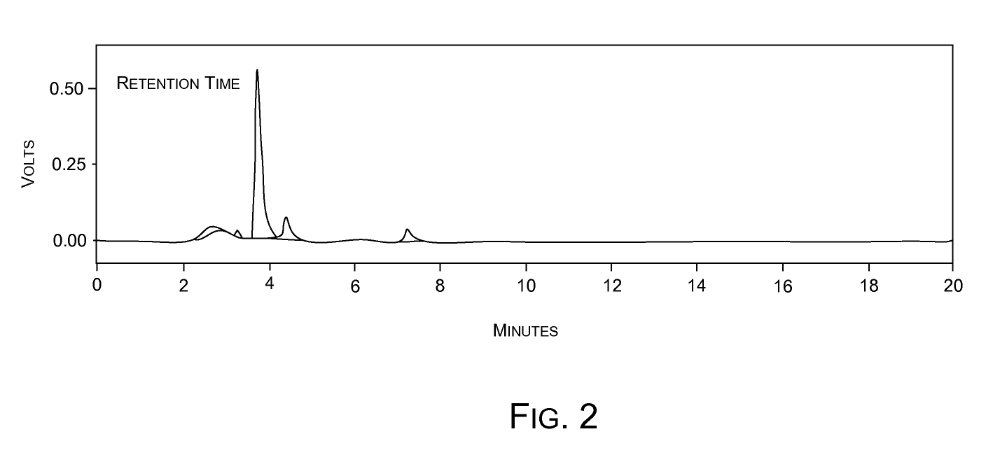

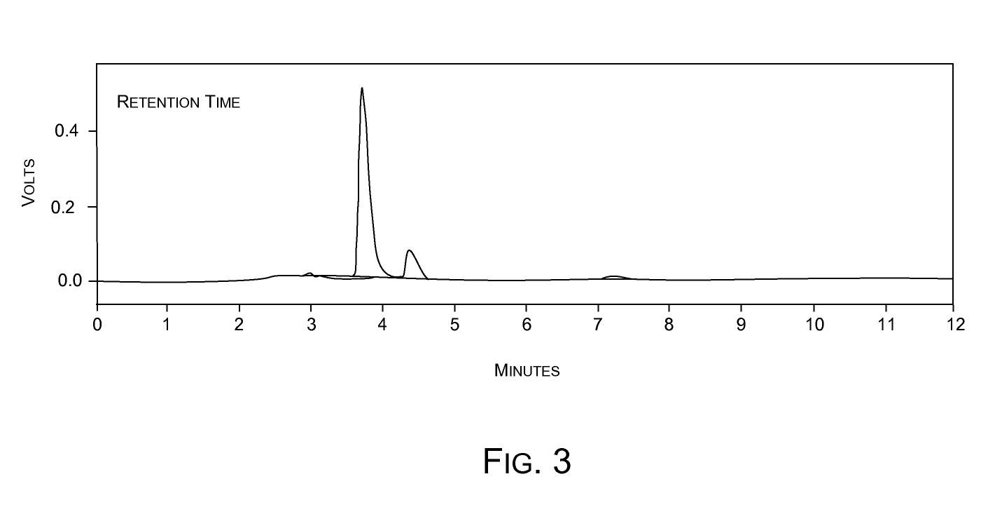

20 ml of the extract obtained using the method explained in example 1 was taken and filtered thrice using whatman filter paper. Thereafter, 20 μL of the extract was injected in the high performance liquid chromatography HPLC injector of a Shimadzu HPLC system equipped with SPD 10A VP UV VIS detector. The column used was a Nova-pak C18. The mobile phase consisted of 0.06% methanol and phosphoric acid in a ratio of 50:50. The detection wave length was 360 nm. The extract was then run in the Nova-pak C18 column for 20 minutes at a flow rate of 1 ml/min. Similarly, an analysis of the extract obtained using the method of example 2 was performed. FIG. 2 illustrates a chromatogram of the extract obtained using the method of example 1. A peak of 7.258 with an area of 550909 milli-absorbance unit (mAU) corresponds to an active ingredient quercetin. FIG. 3 illustrates a chromatogram of the extract obtained using the conventional method in example 2. A peak of 7.228 with an area of 121931 mAU corresponds to the active ingredient quercetin.

Table 1, provides a comparative chart for the extract obtained using the method of example 1 and using the method of example 2. The concentration of quercetin obtained using the method of example 1 is higher than the concentration of quercetin obtained using the method of example 2.

TABLE 1

| | | Volume | | Concentration of |
|---|---|---|---|---|
| | | of | | Active |
| | | Extract | | Ingredient |
| Extraction | Contamination | obtained | % Recovery | (μg/ml) |
| Method | observed | (ml) | of Extract | Quercetin |
| Example 1 | Nil | 850 | 85 | 16.447 |
| Example 2 | After 7 days | 700 | 70 | 3.64 |

Various embodiments of the invention provide a method of extracting a plurality of extracts from Calligonum comosum. The plurality of extracts obtained is easily separated. The method provides one or more sterile liquid extracts as a result of boiling the one or more stalks for a first predefined time duration at a predefined temperature and a predefined pressure. The one or more sterile liquid extracts are therefore free from contamination. The volume of one or more active ingredients present in the one or more sterile liquid extracts are greater when compared to one or more active ingredients present in the extract obtained using conventional methods. The one or more sterile liquid extracts can be used in pharmaceuticals due to the therapeutic properties of the one or more active ingredients. Further, one or more sterile liquid extracts can be used in leather industry for polishing. Also, the one or more solid residue can be used as fodder for animals.

Those skilled in the art will realize that the above recognized advantages and other advantages described herein are merely exemplary and are not meant to be a complete rendering of all of the advantages of the various embodiments of the invention.

In the foregoing specification, specific embodiments of the invention have been described. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of the invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A method for extracting a plurality of extracts from Calligonum sp., the method comprising:
boiling at least one stalk of Calligonum sp. along with at least one liquid in a sterilizing apparatus for a first predefined time duration at a predefined temperature and a predefined pressure;
obtaining a plurality of extracts from the at least one stalk of Calligonum sp., the plurality of extracts comprising at least one sterile liquid extract and at least one sterile residue; and
separating the at least one sterile liquid extract from at least one sterile residue.

2. The method of claim 1, wherein concentration of at least one active ingredient in the at least one sterile liquid extract is greater as compared with extracts of Calligonum sp. obtained by conventional methods of extraction.

3. The method of claim 1, wherein the Calligonum sp. is at least one of a Calligonum comosum, Calligonum crinitum, and Calligonum tetrapteurum.

4. The method of claim 1, wherein the sterilizing apparatus is one of an autoclave, a pressure cooker, a closed container, and a bioreactor.

5. The method of claim 1, wherein the at least one liquid comprises water.

6. The method of claim 1, wherein the at least one stalk is boiled in a predetermined volume of the at least one liquid.

7. The method of claim 6, wherein the predetermined volume ranges from 500 ml to 1500 ml per 0.25 kg to 0.75 kg of the at least one stalk.

8. The method of claim 1, wherein the predefined temperature ranges from 100° C. to 150° C.

9. The method of claim 1, wherein the predefined pressure ranges from 10 psi to 20 psi.

10. The method of claim 1, wherein the first predefined time duration for boiling the at least one stalk ranges from 20 minutes to 40 minutes.

11. The method of claim 1, wherein the at least one active ingredient comprises at least one of a flavanoid.

12. The method of claim 1, wherein the at least one active ingredient comprises a kaempferol, a quercetin, a quercetin-3-0-β-D-glycoside (isoquercitin), a kaempferol-3-0-β-glucuronide, a catechin, a rutin, a procyanidin, a Carotinoid, a violaxanthin, a neoxanthin tannin, a sterol and a triterpene.

13. The method of claim 12, wherein the at least one active ingredient in the at least one sterile liquid extract comprises at least one of an anti-inflammatory, an antioxidant, an antimicrobial, an anticancer, a cardio-protective, a neuro-protective, an antidiabetic, an anti-osteoporotic, an estrogenic/anti-estrogenic, an anxiolytic, an analgesic, an anti-allergic an anti-ulcer or a cytoprotective ingredient.

14. The method of claim 1, wherein the at least one sterile residue is dried at a predefined temperature for a second predefined time duration.

15. The method of claim 14, wherein the predefined temperature for drying the at least one sterile residue ranges from 40° C. to 60° C.

16. The method of claim 14, wherein the second predefined time duration for drying the at least one sterile residue ranges from 24 hours to 96 hours.

17. An extract obtained from Calligonum sp. using a method, the method comprising:
boiling at least one stalk of Calligonum sp. along with at least one liquid in a sterilizing apparatus for a first predefined time duration at a predefined temperature and a predefined pressure; and
obtaining at least one sterile liquid extract from the at least one stalk of Calligonum sp.

18. The extract of claim 17, wherein concentration of at least one active ingredient in the extract is greater as compared with extracts of Calligonum sp, obtained by conventional methods.

19. The extract of claim 17, wherein the Calligonum sp. is at least one of a Calligonum comosum, Calligonum crinitum, and Calligonum tetrapteurum.

20. The extract of claim 17, wherein the at least one active ingredient comprises at least one of a flavanoid.

21. The extract of claim 17, wherein the at least one active ingredient comprises a kaempferol, a quercetin, a quercetin-3-0-β-D-glycoside (isoquercitin), a kaempferol-3-0-β-glucuronide, a catechin, a procyanidin, a rutin, a Carotinoid, a violaxanthin, a neoxanthin tannin, and a sterol or a triterpene.
